(19) United States
(12) Patent Application Publication
(10) Pub. No.: US 2006/0275514 A1
(43) Pub. Date: Dec. 7, 2006

(54) Slimming soap

(76) Inventor: Irene Brigetta Reece, Woodstock, GA (US)

Correspondence Address: IRENE E. REECE, 504 BROOKSDALE DR., WOODSTOCK, GA, 30189, US

(21) Appl. No.: 11/144,086

(22) Filed: Jun. 6, 2005

(51) Int. Cl. A61K 36/886; A61K 36/752

(52) U.S. Cl. 424/736; 424/744

(57) ABSTRACT

A soap designed to help you eliminate waste and increase energy levels with no more effort than that taken to bathe. A slimming soap is comprised of a glycerin soap base of 0.7999333%, Aloe Vera Oil 0.0499958%, Papaya Juice 0.0499958%, Parsley 0.02499979%, Cleavers 0.0249979%, Chickweed 0.0249979%, Comfrey 0.0249979% and Saffron 0.0000838%

This slimming soap is designed to help you lose inches. The prime ingredient is Glycerin Soap Base 0.7999333%. Glycerin is hygroscopic, which means that it absorbs water from the air. As it draws water in, it will moisten and soften the skin. The character of glycerin makes it an excellent carrier of product into the skin.

Papaya, 0.0499958%, is this soap's citrus exfoliant. Its acidic properties will eat away the dead skin cells on the surface of the skin to allow the herbal properties of the soap to permeate without the impedance of surface dirt or dead skin.

Aloe Vera Oil, 0.0499958%, is one of the safest and best, warm and stimulating purgatives to the persons of sedentary habits. An ordinary small dose, as that which is in my soap, takes from 15 to 18 hours produce an effect. Its action is exerted mainly on the large intestine. And its molecular size is readily absorbed through the skin.

Parsley, 0.0249979%, is used in this soap as a detoxificant. This is used chiefly for its diuretic properties. It is very comfortable to the stomach, and has been known to remove obstructions in both the liver and spleen.

Cleavers (Clivers), 0.0249979% is a known diuretic and a tonic

Comfrey, 0.0249979%, is a known demulcent. Demulcent is a gummy substance which produces mucilage that soothes irritations and helps line the intestinal tract.

Chickweed, 0.0249979% is also a demulcent. This herb is good for constipation.

Saffron, 0.0000838%, is used as a mild stimulant. Its carminative properties relieve gas and griping. Its diaphoretic properties are used to induce sweating.

1. (canceled)

2. A slimming soap is comprised of a glycerin soap base of 0.7999333%, Aloe Vera Oil 0.0499958%, Papaya Juice 0.0499958%, Parsley 0.02499979%, Cleavers 0.0249979%, Chickweed 0.0249979%, Comfrey 0.0249979% and Saffron 0.0000838%
